(19) United States
(12) Patent Application Publication
(10) Pub. No.: US 2006/0217613 A1
(43) Pub. Date: Sep. 28, 2006

(54) Method and apparatus for sizing stents

(76) Inventors: Daniel R. Lucas, Anaheim, CA (US); Kosuke Inoue, Corona Del Mar, CA (US)

Correspondence Address: WILLIE KRAWITZ, 3001 CHAPEL HILL ROAD, ORANGE, CA, 92687-1927, US

(21) Appl. No.: 11/079,130

(22) Filed: Mar. 12, 2005

(51) Int. Cl. A61B 5/02

(52) U.S. Cl. 600/481

(57) ABSTRACT

A method and apparatus for sizing stents is provided by employing a thin-walled, compliant, silicone rubber or elastomer balloon catheter into a patient's artery to determine plaque size, contours and plaque length. One or both ends of the catheter may incorporate a radiopaque material to locate the catheter and enable the length and size of the stent to be determined. The catheter may incorporate one or more sensors to determine the density and contours of the plaque. Additionally, one or more relief bores may be incorporated into the catheter to permit blood flow during the short period of time when the artery is blocked, thereby reducing the possibility of oxygen starvation of the heart, brain and other organs.

### BACKGROUND OF THE INVENTION:

This invention relates to the area of percutaneous translumen coronary angioplasty (PTCA), and percutaneous translumen angioplasty (PTA), and to a method and apparatus for enabling a surgeon to select an appropriate stent for insertion into a vein, artery, saphenous vein grafts, perforations, aneurysms, and the like.

It is important when installing a catheter into an artery to determine the size and contours of a stent and that the insertion does not produce excessive pressure which could disrupt or dislodge the plaque. Also, it is important that the insertion itself proceed quickly, say in less than about 90 seconds, and in an efficient manner, without trauma to the patient. Obviously, the contour of the catheter resulting from the insertion should provide a suitable replica of the plaque area into which the stent is subsequently inserted.

Present sizing catheters are generally manufactured of thin-walled, polyurethane material which are non-compliant with respect to the contours of the plaque, as is disclosed in U.S. patent application Ser. Nos. 10/444,234; and 10/655,532. Additionally, current diagnostic angiographic techniques lack the precision to define the plaque size, especially in diabetics where the plaque lesion is diffuse.

In addition, it is difficult to optimally deploy the stent to cover an entire lesion, and this leads to slipping of the stent, restenosis and other complications.

Using a technique known as IVUS, a tiny ultrasound probe is threaded into a body vessel, and this technique been used to obtain a cross sectional view of the plaque. But this technique is cumbersome, interrupts blood flow, and can produce laceration of a vulnerable plaque, besides being costly.

In “THE METRICACTH SYSTEM” sold by Medventures, a balloon catheter is inflated in say an artery at the entrance to a plaque area, and the extent of inflation indicates the volume of a stent which needs to be adjusted in the artery prior to inserting the into the plaque area. However, this procedure does not address the manner in which a plaque contour is obtained, plaque structure, or size of the plaque itself.

Multilayered, cured silicone catheters are described in U.S. Pat. Nos. 5,762,996 (1998) and 5,795,332 (1998), both to the inventor Daniel R. Lucas, herein. These two patents disclose the manufacture and use of these catheters, but do not suggest using these catheters in a plaque analysis environment. The use of the catheters proposed by these patents include removal of thrombus (clots), gallstones and emboli.

### THE INVENTION

According to the invention, a cured silicone catheter is provided suitable for insertion into an artery, and the like which when inflated, conforms to the contours and size of the plaque. Inflation of the catheter produces a contour of the plaque area and in conjunction with catheter markers can also enable a determination of plaque length.

When the inflated catheter which has been conformed to the contours of a plaque area is displayed by means of x-ray techniques, a surgeon will be able to better determine catheter length, the nature of the contours, and possibly the type of plaque to be encountered. Hence, not only can the proper selection of a stent be determined, but also when installing the stent into a patient, advance knowledge of the type of plaque which might be encountered; this will enable the surgeon to conduct the procedure in a timely fashion. This in turn will reduce the possibility of plaque rupture, and related problems.

Moreover, additional various features can be built into the catheter which can improve its function, and such function includes the use of micro sensors to determine the depth and pliability of the plaque.

Another feature of this invention includes the use of relief bores which permit a small flow of blood to occur during the period when the vein or artery is blocked off during the time measurement and contour determination is taking place.

### IN THE DRAWINGS

FIG. 1 is a cross sectional and partial perspective view of a heart showing the general location where a catheter of this invention is installed for measurement purposes, and shown in longitudinal cross section;

FIG. 2 is a view in longitudinal section showing the catheter of this invention expanded against plaque material for determine contour evaluation of the plaque;

FIG. 3 is a view in longitudinal section showing the catheter as it is withdrawn from the plaque area;

FIG. 4 is a longitudinal view in side elevation showing blood drainage bores incorporated into the catheter and which permit drainage of blood during a catheter measurement procedure;

FIG. 5 is the same view as FIG. 4 when inserted into a plaque area in an artery;

FIG. 6 is a view taken along A-A of FIG. 5, and,

FIG. 7 is a longitudinal view in axial section showing the location of micro sensors in the catheter for determining plaque density and other characteristics.

### DESCRIPTION OF THE PREFERRED EMBODIMENTS

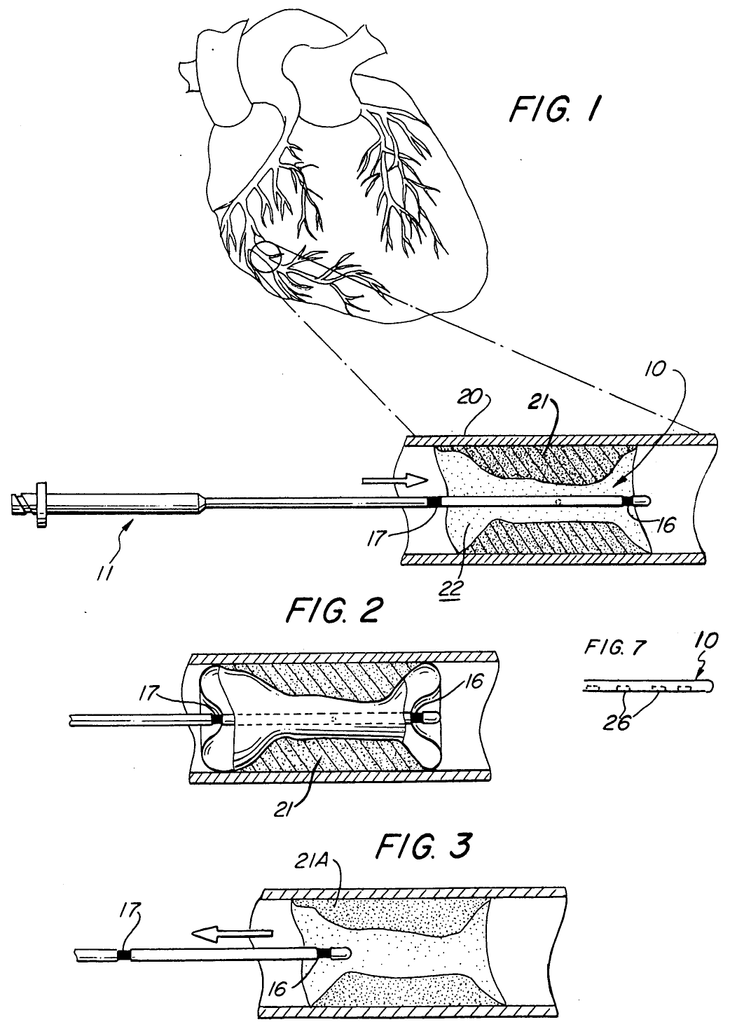

The catheter 10 of this invention is shown in FIGS. 1-3 and comprises a cured silicone rubber or elastomer formed typically by the process described in U.S. Pat. Nos. 5,762,996 and 5,795,332 supra, and incorporated herein, by reference. Preferably, the viscosity of the silicone and solvent (e.g., isopropanol, xylene, etc.) is adjusted prior to the manufacture of the catheter to control softness of the catheter.

Contours of the plaque can be determined by Quantitative Coronary Angiography (QCA), as described in the article, “EVALUATION OF CENTER-LINE EXTRACTION ALGORITHMS IN QUANTITATIVE CORONARY ANGIOGRAPHY”, by H. Greenspan, et al, IEEE Transactions on Medical Imaging, Vol. 20, No. 9, Sep. 2001. A suitable software program, CATHLAB, may be utilized for this purpose.

When a balloon catheter of this invention is inserted into a plaque area of say an artery, its thin, compliant structure will avoid significant structural damage to the plaque formation upon insertion. Also upon inflation, the catheter will reduce damage to the plaque, particularly when used in conjunction with micro chip sensors which are described, infra.

Equally important, when expanded by a radiopaque material, the compliant nature of the catheter (unlike a polyurethane film), will conform to the contours of the plaque and provide better information on the plaque structure using QCA techniques described, supra.

The catheter shown in FIGS. 1-3 comprises a cured silicone rubber or elastomer materials formed typically by the process described in U.S. Pat. Nos. 5,762,996 and 5,795,332 supra. Additionally, these silicone materials can be placed on thermoplastic materials such as polyurethanes and polyether block amides (POBAXR). Preferably, the viscosity of the silicone and solvent (e.g., isopropanol, xylene, etc.) is adjusted prior to the manufacture of the catheter to control catheter softness. Typical catheter sizes vary from about 25 mils-90 mils in diameter, and a balloon wall thickness of about 3 mils-12 mils.

As shown in FIGS. 1-3, an artery wall 20 is shown enclosing a contoured plaque area 21 which in turn defines a narrowing passage 22 of the artery. The catheter 10 is shown being inserted into the passage 22 using say a Seldinger introducer 11. FIG. 3 shows the catheter following complete introduction into passage 22 of the artery, prior to inflation.

FIG. 3 illustrates retraction of the catheter 10 from the plaque area 21, and the structure 21A illustrates that the plaque has undergone reduced compression during the analysis procedure.

Generally, a plaque analysis procedure should take place within about ninety (90) seconds, otherwise the blockage of blood flow may well cause serious injury to vital organs of a patient due to lack of blood carrying oxygen. Hence, drainage of blood which carries dissolved oxygen through the drainage bores should somewhat alleviate this type of problem.

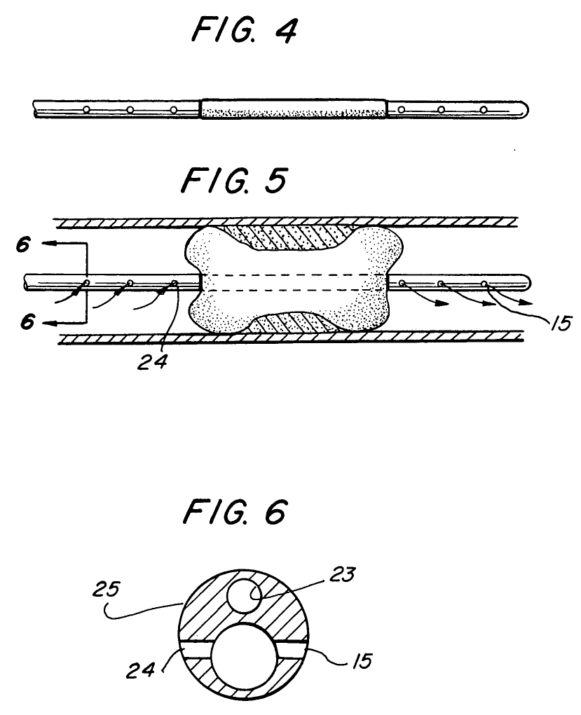

To achieve this purpose, another embodiment of this invention is shown in FIGS. 4-6, and illustrates a double lumen catheter 25 useful for providing blood flow by employing blood drainage bores from the catheter to the patient during an analysis procedure. As shown in FIGS. 4-6, multiple blood drainage bores 15 in the catheter enable a small amount of blood to flow through the catheter when inserted into a plaque area. Suitable drainage bores would be about 10-20 mils in diameter, and about 1-20 bores could be utilized. As shown in FIG. 6, air inflation for the balloon is provided through a bore 23, while blood inlet bores 24 and blood drainage bores 15 permit blood flow to be supplied to the patient.

To establish the end points of the catheter which can determine a suitable length of a subsequently inserted stent, another embodiment of this invention utilizes radiopaque end markers 16, 17 for the catheter. These radiopaque end markers are adjusted by visual approximation to extend somewhat beyond the immediate plaque area. The end markers may also be indexed for suitable visual reference, and if desired, one or both end markers may be employed. Using a radiopaque material for inflation, an x-ray display of the markers is produced to determine the appropriate length of the catheter.

This is accomplished by manipulating the catheter 10 adjacent the plaque so that the radiopaque end markers 16, 17 are extended (by visual approximation) beyond the immediate boundaries of the plaque as shown in the x-ray display. This manipulation may be assisted by using indexing notches on the end markers for this purpose. Since undetected plaque material may extend beyond the immediate plaque area shown in the x-ray, this technique will reduce the possibility that the stent size length will be too short. Following deflation (FIG. 5), the catheter 10 is then removed, and the ends are cut off, yielding the appropriate length which a surgeon would then utilize to size the stent, without having to make manual and time consuming measurements.

Another embodiment of this invention is described in FIG. 7 in which one or more micro chip sensors 26 are shown implanted into the catheter 10. These sensors may be used during expansion of the balloon and would yield information of the type of plaque such as hard, medium, soft, thickness, contours in conjunction with QCA analysis, length, etc. This in turn will enable the type and size of stent which should be utilized by a surgeon.

It will be apparent that various combinations of the invention components are possible, in addition to the basic aspect of utilizing a compliant thin-walled catheter to determine the contour structure and length of plaque, such as plaque density and plaque length. In addition, various combinations of radiopaque inflation may be utilized, such as inflation with a radiopaque material; incorporating a radiopaque material into the catheter itself; utilizing air inflation of a multi lumen catheter which incorporates a radiopaque material, etc.

The catheter of this invention enables an improved determination of plaque contour, as well as an improved method for determining plaque length, which is usually measured by a surgeon, with its attendant inaccuracies, and an excessive consumption of time.

1. A balloon catheter manufactured of a cured silicone elastomer or rubber, for insertion into an artery, and the like of a patient which encloses a plaque material, the catheter when inflated being compliant with the contours of the plaque, with reduced damage to the plaque.

2. The catheter of claim 1, providing end markers for determining end areas of the plaque, and upon withdrawal from the artery, the catheter is adapted to be cut at each end, thereby providing a an accurate length size and contour type for a timely selection of a suitable stent type, thereby enabling insertion adjacent to, and securement of the plaque.

3. The balloon catheter of claim 1, including micro chip sensors adapted to analyze the type and thickness of the plaque.

4. The balloon catheter of claim 2, including micro chip sensors adapted to analyze the type and thickness of the plaque.

5. The balloon catheter of claim 1, including a dual lumen catheter defining an inflation bore to the catheter and blood drainage bores to the patient.

6. The balloon catheter of claim 2, including a dual lumen catheter defining an inflation bore to the catheter, and blood drainage bores to the patient.

7. The balloon catheter of claim 3, including a dual lumen catheter defining an inflation bore to the catheter, and blood drainage bores-to the patient.

8. A balloon catheter suitable for insertion in an artery, and the like, of a patient, and adjacent a plaque area of the artery, the catheter including one or more micro chip sensors adapted to detect one or more of: length, thickness, structure and contours of the plaque.

9. The balloon catheter of claim 5, in which the catheter is constructed of a cured silicone elastomer or rubber.

10. The balloon catheter of claim 5, including a dual lumen catheter defining inflation and blood drainage bores to the patient.

11. The balloon catheter of claim 5, including end markers defined on the catheter for determining the catheter length adjacent the plaque.

12. The balloon catheter of claim 8, including end markers defined on the catheter for determining the catheter length adjacent the plaque.

13. A method of sizing a stent for insertion into an artery, and the like, for expansion into an area of plaque material, the method comprising inserting a compliant, cured silicone rubber or elastomer balloon catheter into the plaque area, expanding the balloon against the plaque material, thereby determining plaque contour and effective length of the plaque material without substantial damage to the plaque, withdrawing the catheter, and sizing the stent depending on the aforesaid determination.

14. The method of claim 13, incorporating micro sensors into the catheter to determine one or more of: structure, thickness, length, and contours of the plaque.

15. The method of claim 13, providing a dual lumen catheter defining inflation bores, blood supply bores and blood drainage bores to a patient.

16. The method of claim 13, providing end markers defined on the catheter for determining the effective plaque length adjacent the end markers.

17. The method of claim 13, comprising inserting a compliant, cured silicone elastomer or rubber balloon catheter into the plaque area, expanding the balloon against the plaque material, thereby determining plaque contours and effective length of the plaque material without substantial damage to the plaque, withdrawing the catheter, and sizing the stent depending on the aforesaid determination, the catheter including an end marker disposed on at least one end of the balloon to determine the effective length of the plaque material.

18. The method of claim 17, including incorporating into the balloon one or more micro sensors to detect on or more of: length, thickness, structure, and contours of the plaque.

19. The method of claim 17, comprising a dual lumen catheter defining an expansion bore, and blood inlet bores to the catheter and blood outlet bores from the catheter to a patient.
